(19) United States
(12) Patent Application Publication
(10) Pub. No.: US 2012/0267253 A1
(43) Pub. Date: Oct. 25, 2012

(54) STERILIZATION METHOD AND APPARATUS FOR MEDICAL INSTRUMENTS COMPLYING WITH HIGH-LEVEL DISINFECTION STERILIZATION STANDARDS

(76) Inventor: Chil-Young Kim, Gyeonggi-do (KR)

(21) Appl. No.: 13/518,403

(22) PCT Filed: Dec. 14, 2010
(86) PCT No.: PCT/KR10/08933

§ 371 Date: Jun. 22, 2012

(30) Foreign Application Priority Data
Dec. 23, 2009 (KR) 10-2009-0129372

(51) Int. Cl. A61L 2/18; C25B 1/26; C25B 1/13; C25B 1/30; C25B 9/00

(52) U.S. Cl. 205/412; 205/466; 204/274

(57) ABSTRACT

Provided is a sterilization method and apparatus for medical instruments. In the method, a solution containing chlorine and having a temperature of about 60° C. or more is prepared. An electrode is disposed in a container containing the solution and the medical instrument is immersed in the solution such that the medical instrument is disposed over the electrode. The solution is electrolyzed by applying a current to the electrode to generate sterilizing components of free chlorine comprising hypochlorous acid, hydrogen peroxide (H₂O₂), OH radical, and ozone (O₃) and sterilize the medical instrument by the components which move up in the opposite direction of gravity from the electrode.

### TECHNICAL FIELD

The following disclosure relates to a sterilization method for medical instruments or devices or equipments (hereinafter referred to simply as ‘instruments’), and in particularly, to a sterilization method for medical instruments, which enables to sterilize complying with the strict High-Level Disinfection (HLD) standards within a shorter time and allows medical instruments to be immediately reusable because residues irritating a human body do not remain during the sterilization.

### BACKGROUND ART

Recently, as the contamination of air and soil gets severe, environmental diseases such as allergy and atopy are increasing. Also, interest in health is increasing day by day along with the increase of interest in well-being. Accordingly, harmless methods for washing and effectively sterilizing reusable medical instruments such as endoscope apparatuses inserted into human organs for examination, catheters inserted into blood vessels, trocar, and surgical instruments have been studied.

Generally, reusable medical instruments or patient-caring instruments are sterilized before each time of use, and are used in a form of insertion into tissues or blood vessels of a human body. As typical sterilization methods that are performed at hospitals, there are sterilization methods in which instruments are allowed to be exposed to a hot steam or an ethylene oxide gas, or to be thermally dried. However, these methods are difficult to apply to the medical instruments that are sensitive to heat. Accordingly, sterilization method using original or diluted chemicals are being applied to the heat sensitive instruments, but there is a limitation in that the sterilization state is insufficient and they can be harmful to the human body even though pathogenic bacteria can be sterilized.

Above all, although not yet adopted in Korea, U.S. FDA regulates the High Level Disinfection (HLD) standards to ensure safety of patient from second infection, and allows medical instruments such as endoscopes sensitive to the human body to be sterilized according to the HLD standards.

The HLD standards require all microorganisms or pathogenic bacteria to be eliminated by 6 log₁₀. When sterilized by exposure to hot steam or boiling water, some viruses such as HBV, HCV, and HIV or bacteria may not be completely sterilized, and also the sterilization takes about 20 to 40 minutes, making it practically difficult to perform sterilization complying with the HLD standards on frequently and repeatedly re-used medical instruments such as endoscope or other medical instruments. Thus, as medical instruments that are not sufficiently sterilized are repetitively reused, subjects may be exposed to the risk of secondary infection.

A process of sterilizing medical instruments using chemicals such as formaldehyde, chlorine gas, or glutaraldehyde in compliance with the HLD standard has a limitation in which metallic parts of the medical instruments can be corroded at a concentration of about 0.5% or more. Also, since the medical instruments need to be immersed in chemicals for 20 minutes or more, the process is inappropriate for sterilization of medical instruments that are used frequently and repeatedly.

Accordingly, a method for sterilizing frequently and repeatedly-reused medical instruments complying with the strict HLD standards within a shorter time of about 2 or 3 minutes, not typical 20-30 minutes is desperately needed.

### DISCLOSURE

### Technical Problem

Accordingly, the present disclosure provides a sterilization method for medical instruments, which enables to sterilize complying with the strict High Level Disinfection (HLD) standards within a shorter time of two to three minutes and allows medical instruments to be immediately reusable because residues irritating a human body do not remain during the sterilization.

The present disclosure also provides a sterilization method for medical instruments, which can effectively eliminate pathogenic bacteria that remain on a medical instrument and cause secondary infection before the use of the medical instruments that are repeatedly and frequently reused, by performing sterilization complying with the strict HLD standards on the medical instrument within a shorter time, and thus minimize the sterilization time for reuse of medical instruments such as an endoscope, a catheter inserted into a vessel, a trocar, and a surgery instrument and simultaneously prevent secondary infection.

The present disclosure also provides a sterilization method for medical instruments, which can fundamentally prevent side effects caused by the sterilization of the medical instrument by allowing a sterilization liquid not to irritate the mucous membranes of patient's lung, eyes, and nose, and patient's skin even when the sterilization liquid is not completely removed after the sterilization of a medical instrument and the medical instrument is immediately used.

### Technical Solution

In one general aspect, a sterilization method for medical instruments, including: preparing a solution containing chlorine and having a temperature of about 60° C. or more; disposing an electrode in a container containing the solution and immersing the medical instrument in the solution such that the medical instrument is disposed over the electrode; and electrolyzing the solution by applying a current to the electrode to generate sterilizing components of free chlorine comprising hypochlorous acid (HOCl), hydrogen peroxide (H₂O₂), OH radical, and ozone (O₃) and sterilize the medical instrument by the sterilizing components which move up in the opposite direction of gravity from the electrode.

In order to meet the High Level Disinfection (HLD) standards, regarding a limitation in which mycobacteria such as tubercular bacillus and Hansen's bacillus surrounded by a strong fat layer cell wall cannot be eliminated because hot water or germicide cannot infiltrate into them, the strong cell wall of the fat layer may be deformed using disclosure, and free chlorine such as hypochlorous acid (HOCl) that are generated by maintaining a solution containing free chlorine such as hypochlorous acid having a strong sterilizing efficacy at a temperature of about 66.9° C. and electrolyzing the solution containing chlorine. Sterilizing components such as free chlorine including hypochlorous acid, hydrogen peroxide (H₂O₂), OH radical, and ozone (O₃) generated in saline water can infiltrate into the cell wall to eliminate most mycobacteria such as tubercular bacillus and Hansen's bacillus within a shorter time of about 2 minutes to about 3 minutes.

In the sterilizing of the medical instrument, a large amount of hypochlorous acid (HOCl) may be generated using chlorine in a solution, and simultaneously, the amount of intermediate sterilizing components of hydrogen peroxide (H₂O₂), OH radical, and ozone (O₃) which have immediate high instantaneous sterilizing efficacy may be maximally generated by applying a direct current of about 30 mA to about 2,000 mA to electrodes coated with platinum when the electrodes are spaced from each other by about 1 mm to about 3 mm. Thus, mycobacteria stuck on the medical instrument can be eliminated by 6 log₁₀ within a shorter time of about two or three minutes in compliance with the HLD standards.

In this regard, the test results will be described below.

### Embodiment 1

### Test of Viability of Mycobacteria According to Exposure Time at Temperature of 60° C. or More

Viability of bacteria were tested by exposure time under a condition that M. tuberculosis were exposed to free chlorine at temperatures of 60° C., 65° C., 70° C., and 75° C., respectively. First, a solution of Macfarland No II (about 6*108/ml) was manufactured with Mycobacterium tuberculosis. Thereafter, a 0.3% NaCl solution was prepared with sterile distilled water, and then a 0.3% saline solution was put into a water tank at temperature conditions of 60° C., 65° C., 70° C., 75° C., and 85° C., respectively. Next, a 0.3% saline solution of 45 ml and a bacteria solution of 5 ml were mixed, and then a 120 mA current was applied to electrodes at each temperature to evaluate the viability of bacteria according to the exposure time. The test results are shown as follows.

| | 60° C. | 65° C. | 70° C. | 75° C. | 85° C. |
|---|---|---|---|---|---|
| | Log | Log | Log | Log | Log |
| | reduction | reduction | reduction | reduction | reduction |
| 30 sec | 2.11 | 2.43 | 2.60 | 3.52 | 3.60 |
| 60 sec | 3.62 | 3.82 | 3.98 | 4.20 | 4.30 |
| 120 sec | 6.00 | 6.00 | 6.00 | 6.00 | 6.00 |

For reference, since an apparatus used to measure a viable population of bacteria used in the test is measurable up to 6 log₁₀, the log reduction marked as 6.00 in the table indicates a sterilizing efficacy higher than 6 log₁₀.

### Comparison Example 1

### Test on Viability of Mycobacteria According to Exposure Time at Room Temperature

Viability of bacteria were tested by exposure time under a condition that M. tuberculosis were exposed to free chlorine at a room temperature. First, a solution of Macfarland No II (about 6*108/ml) was manufactured with Mycobacterium tuberculosis. Thereafter, a 0.3% NaCl solution was prepared with sterile distilled water, and then a 0.3% saline solution was put into a water tank at room temperature condition. Next, a 0.3% saline solution of 45 ml and a bacteria solution of 5 ml were mixed, and then a 120 mA current was applied to electrodes at room temperature to evaluate the viability of bacteria according to the exposure time. The test results are shown as follows.

| | Room temperature |
|---|---|
| | Log Reduction |
| 30 sec | N/A |
| 60 sec | 1.9 |
| 90 sec | 2.2 |
| 120 sec | 2.3 |

At room temperature, the initial population of bacteria was maintained until about 30 seconds lapsed, but the population was reduced while 60 seconds was passing. Thereafter, even though sufficient time passed, the population was rarely reduced. Accordingly, it can be concluded that it is hard to meet the HLD standards even after sterilization is performed for a sufficiently long time.

Meanwhile, after the sterilizing of the medical instrument, discharging the solution and secondarily sterilizing the medical instrument by performing electrolysis such that a low concentration of free chlorine including HOCl less than 6 ppm concentration on the electrodes may be further included.

Thus, since the medical instrument is rinsed and washed by free chlorine including HOCl of about 6 ppm or less that does not irritate the mucous membranes of a human body, a high concentration of HOCl that may be harmful to the mucous membranes of the human body may not remain on the medical instrument. Accordingly, by sterilizing and washing the medical instrument through the secondary sterilization process for a short time of about 10 seconds to about 30 seconds after the sterilizing of the medical instrument, although free chlorine remaining on the surface of the medical instrument after the sterilizing of the medical instrument is introduced into the body of a subject, the subject may not feel irritated or uncomfortable even when the medical instrument is used immediately after the sterilization.

In this case, secondary sterilizing of the medical instrument such that a low concentration of free chlorine including HOCl of about 6 ppm or less is disclosed in Korean Patent Publication No. 2009-19639, filed and owned by the present applicant, which is incorporated into the present disclosure as a part.

Also, the sterilizing of the medical instrument may be performed by maintaining the saline water at a temperature of about 92.9° C. or more. As shown in Table 1, the cell wall of a fat layer formed on the surface of mycobacteria such as tubercular bacillus or Hansen's bacillus are usually changed into an infiltrative state at a temperature of about 66.9° C. However, in case of M. terrae among mycobacteria, free chlorine of saline water cannot infiltrate into the cell wall thereof until the temperature of the fat layer in the surface thereof reaches about 92.9° C. Accordingly, the temperature of the saline water may be maintained at a temperature of about 92.9 or more in order to eliminate all M. terrae within a shorter time.

TABLE 1

| | | Fat layer Maximum |
|---|---|---|
| | Size | deformation |
| Type of Mycobacteria | (Total number of carbon) | temperature (° C.) |
| C. bovis | 32 | 35.9 |
| C. pseudotuberculosis | 38 | 39.4 |
| M. vaccae | 74 | 59.3 |
| M. aurum | 76 | 60.3 |
| M. chelonae | 77 | 62 |
| M. tuberculosis H37Rv | 80 | 62.8 |
| M. smegmatis mc2-6 | 77 | 63.5 |
| M. smegmatis mc2-155 | 77 | 64.0 |
| M. avium | 80 | 66.9 |
| M. terrae | 79 | 92.9 |

Also, with only a small amount of free chlorine could have a high treatment effect by maximizing the composition of HOCl with high sterilizing efficacy among other residual chlorines generated by the electrolysis from at least 50% to almost 100%, through sterilizing the medical instrument with slightly acid or neutral saline water of PH 4.0 to 9.0. Also, since sterilization solution manufactured at the same time has an acid degree that the sterilization solution does not irritate the mucous membranes of eyes and nose of a human body, although the sterilization solution remains after the washing of the medical instrument, patients who contact the medical instrument are not irritated.

Particularly, since the acid degree of tap water or underground water that are easily available is slightly acid or neutral, physiological saline may be manufactured by mixing an appropriate amount of salt with tap water that is easily available. Therefore, it is viable to produce sterilization solution in a simple way that sterilizing and cleansing medical instrument can be done simply using inexpensive tap water or underground water.

Moreover, information published by U.S. Environmental Protection Agency in January 1994 recommends that the amount of free chlorine absorbed into the human body should not exceed more than 6 ppm per day based on an adult having a weight of about 70 kg. Since the medical instrument is secondarily washed by a solution having a low concentration below 6 ppm, the medical instrument may be harmless to the human body even when a small amount of solution remains after use. Accordingly, the medical instrument such as an endoscope can be immediately reused without waiting until bactericide evaporates after the sterilization.

In this case, according to parts of the human body to which the medical instrument is applied, it is possible to use saline water having a salt concentration of about 0.3% to about 3% instead of a physiological saline that is a level of an isotonic solution having a salt concentration of about 0.7% to about 1.5%. Although the sterilization solution still remaining on the medical instrument after the sterilization process is absorbed into the human body, since the medical instrument is sterilized by the sterilization solution with a salinity similar to the salinity of the human body, a negative reaction of the human body can be minimized.

In the secondary sterilizing of the medical instrument while controlling the concentration of free chlorine into about 6 ppm or less, when an interval between electrodes is smaller than about 1 mm, the magnitude of current flowing between electrodes may excessively increase, and the flow of gases generated from the electrolysis may not be smooth. Accordingly, a large amount of free chlorine may be generated, or a smaller amount of free chlorine may be generated, making it difficult to uniformly generate free chlorine. When the gap between electrodes is greater than about 3 mm, a high voltage needs to be applied in order to flow current between electrodes. In this case, a current necessarily increases rapidly depending on an overpotential between electrodes (see FIG. 4), which makes it difficult to flow a low current between electrodes and thus generate free chlorine with a low concentration. That is, the gap between electrodes may be maintained from about 1 mm through about 3 mm in order to uniformly maintain a current between electrodes and electron movement in the solution.

In a state where the gap between electrode is maintained from about 1 mm through about 3 mm, if a direct current of about 2.4 V to about 3.3 V is applied, then a low current of about 30 mA to about 200 mA flows between electrodes. In other words, when a low voltage lower than about 2.4 V is applied to electrode, a voltage difference does not occur to a degree that resistance of physiological saline between electrodes is overcome. Accordingly, no current flows between electrodes. When a direct current higher than 3.3V is applied, a rapidly increased current flows between electrodes, making it difficult to uniformly maintain a current value. Since the concentration increase of free chlorine rapidly increases, it is difficult to uniformly generate free chlorine with a low concentration.

In this regard, a forward current i flowing into an external circuit through one electrode is a difference ia-ic between of two currents, an oxidation current ia and a reduction current ic. More specifically, when the magnitude of the overpotential is very small, a forward current by the Butler-Volmer equation is in direct proportion to the overpotential. However, when the magnitude of the overpotential is greater than a certain value, the current exponentially (see FIG. 4). A current flowing in the saline solution with a low salinity concentration of about 0.3% to about 3% including about a 0.9% physiological saline is greatly dependant on an applied voltage and resistance thereof. Accordingly, when the saline water serves as a resistor between the electrodes with flat plates facing each other, a current does not flow between the electrodes regarding a voltage smaller than about 2.4 V, but a rapidly high current flows between electrodes regarding a voltage greater than about 3.3 V flows between electrodes, producing a large amount of free chlorine and thus making it difficult to control at a lower concentration.

Accordingly, when an appropriate voltage is applied and a lower current flows, although carrying proper amount of voltage yet maintaining applying of its low-current voltage then residual chlorine at low concentration between 0.17 ppm and 6.0 ppm can be produced with under stable control. At this point, the current direction given to the electrode should be occasionally switched about every 20 seconds or 2 minutes. Note that if voltage is kept stably during the switching process then the value of current would rapidly be higher that it would cause an issue of not producing residual chlorine such as HOCl at a stable speed. Therefore, when producing low-concentration residual chlorine with more credibility it is more efficient to carry the stably maintained current rather than to maintain voltage when it comes to the power carried to the electrode.

Also, washing the medical instrument by heating the saline water with temperature of 66.9° C. or above containing the residual chlorine produced from the above process would make mycobacteria such as Hansen's bacillus or tubercular, known for its difficulties in sterilization, disinfected rapidly within very short amount of time.

At this point, different from the traditional method, having sterilizing medical instrument through electrode for very short amount of time, 2-3 minutes, minimizes degree of corrosion even when washing metal made medical instruments such as stainless material, and at the same time disinfectant could be done meeting the HDL standard.

On the other hand, regarding production of water including chlorine into a sterilization solution, a production reaction of oxidants such as ozone (O₃), hydrogen peroxide (H₂O₂), OH radical, free chlorine such as HOCl may be performed by the following processes (1) through (5).

(1) The generation of ozone (O₃) starts from electrolysis of water and is completed by a process in which 0 and O₂ are finally combined.

*H₂O-->H⁺+(OH)_{ads}+e⁻

(OH)_{ads}-->(O)_{ads}+H⁺+e⁻

2(OH)_{ads}-->O₂+2H⁺+2e⁻

2(O)_{ads}-->O₂

(O)_{ads}+O₂-->O₃

(2) Hydrogen peroxide is generated by a direct path of electrolysis of oxygen and an indirect path in which OH radicals that is an intermediate product generated by ozonolysis are combined as follows:

Direct Path

O₂+e−-->O₂.⁻

O₂+2H⁺+2e⁻-->H₂O₂

Indirect Path

OH.+OH.-->H₂O₂

(3) HOCl is generated by combination Cl⁻ ions existing in the water into Cl2 and then reaction with H2O as follows:

2Cl−-->Cl₂+2e⁻

2H₂O+2e−-->H₂+2OH⁻

Cl₂+H₂O-->HOCl+H⁺+Cl⁻

(4) Since OH radicals are instantaneously generated and disappear, they cannot be directly measured. However, when ozone exists in the water, ozone reacts with OH⁻ or HO²⁻ that is a conjugate base of H₂O₂ to form a radical chain cycle and finally OH radical.

O₃+OH-->Radical Chain Reaction-->OH.

O₃+HO²⁻ (Conjugate Base of H₂O₂)-->Radical Chain Reaction-->OH.

(5) Microorganisms and microorganics that exist in the water are inactivated or removed by generated oxidants. The microorganisms may be removed by electrosorption, and the microorganics may be removed by reaction with e−, direct electrolysis reaction.

Regarding Microorganism,

M (Microorganism)-->Electrosorption-->Inactivation

Also,

M (Microorganism)+O₃-->Inactivation

M+OH.-->Inactivation

M+HOCl-->Inactivation.

Regarding Microorganics,

M (Microorganics)+e−-->M−

Also,

M (Microorganics)+O₃-->Product

M+OH.-->Product

M+HOCl-->Product

It is can be understood that during the electrolysis, oxidation and sterilization are smoothly performed by oxidants (O₃, H₂O₂, HOCl, OCl⁻, and OH radical) including residual chlorine (HOCl, OCl⁻) generated in the processes (1) to (5), and simultaneously proteins on medical instruments can be effectively removed.

Meanwhile, when a medical instrument to be sterilized is tightly gripped by a specific gripper, the medical instrument may have a little chance of contacting oxidants (O₃, H₂O₂, HOCl, OCl⁻, and OH radical) generated from an electrode, failing to sterilize mycobacteria in compliance with the HLD standards. Accordingly, a forcible flow may be applied in the container such that the whole surface of the medical instrument contacts the solution to allow oxidants to sufficiently contact the medical instrument to be sterilized. Thus, the medical instrument held in the container may be rocked by the flow to allow the whole surface area to contact the oxidants, thus enabling sterilization complying with HLD within a shorter time of about 2 minutes or about 3 minutes.

Although water containing chlorine can be directly heated, in order to easily facilitate the concentration control of a chlorine component, the preparing of the solution may be divided into heating water to a temperature of about 60° C. to about 95° C. and mixing a saline component with the heated water.

Also, the electrode may be coated with platinum. When the platinum coated layer is consumed and worn out according to the use of the electrode to become thinner than a predetermined thickness, shutting off the supplying of the current to the electrode may be additionally included. This is because when the thickness of platinum is not sufficient, the efficiency of the electrolysis is reduced and thus oxidants such as O3, H2O2, HOCl, OCl⁻, and OH radical are not sufficiently generated, failing to sterilize the medical instrument in compliance with HLD. Accordingly, when the number of use increases such that a sufficient amount of oxidant complying with HLD can be generated, a switch on a power source line applying power from the power supply unit to the electrode may be automatically turned off, ensuring reliable sterilization performance of the medical instrument.

The chlorine component of the solution may be supplied from salt.

In the sterilization step, the electrolysis may be performed while maintaining the solution at a temperature of about 60° C. to more effectively remove a fat layer of mycobacteria.

According to another embodiment, the present disclosure provides a sterilization apparatus for medical instruments. The sterilization apparatus may include: a heater for heating a liquid to a temperature of about 60° C. or more; a container containing a solution containing the liquid with chlorine and receiving the medical instrument to be sterilized in the solution thereof; a power supply unit for supplying a direct current; an electrode disposed under the medical instrument so as to be immersed in the solution, electrolyzing the solution using a direct current from the power supply unit to generate sterilizing components of free chlorine including hypochlorous acid (HOCl), hydrogen peroxide (H₂O₂), OH radical, and ozone (O₃), and sterilizing the medical instrument by the sterilizing components which move up in the opposite direction of gravity from the electrode.

Here, the heater is separate from the container while heating water. The heated water may also be supplied to the container. On the other hand, the heater may be configured to heat the heater. In this case, the heater may maintain the container at a temperature of about 60° C. or more.

A part of the medical instrument to be sterilized may be fully immersed in the solution.

Oxidants generated in the electrode move in the opposite direction of gravity. Accordingly, in order to allow more oxidants to contact the surface of the medical instrument, the container may be configured such that the medical instrument can be laid in the container. The electrode may be disposed under the medical instrument that is laid.

### Advantageous Effects

As described above, the present disclosure provides a sterilization method for medical instruments, comprising: preparing a solution containing chlorine and having a temperature of about 60° C. or more; disposing an electrode in a container containing the solution and immersing the medical instrument in the solution such that the medical instrument is disposed over the electrode; and electrolyzing the solution by applying a current to the electrode to generate sterilizing components such as free chlorine including hypochlorous acid (HOCl), hydrogen peroxide (H₂O₂), OH radical, and ozone (O₃) and thus sterilize the medical instrument by the sterilizing components which move up in the opposite direction of gravity from the electrode. Thus, a strong cell wall of a fat layer can be deformed using disclosure, and free chlorine such as hypochlorous acid that are generated by maintaining a solution containing free chlorine such as hypochlorous acid having a strong sterilizing efficacy at a temperature of about 66.9° C. and electrolyzing the solution containing chlorine. Sterilizing components including free chlorine having hypochlorous acid (HOCl), hydrogen peroxide (H₂O₂), OH radical, and ozone (O₃) generated in saline water can infiltrate into the cell wall to eliminate most mycobacteria such as tubercular bacillus and Hansen's bacillus within a shorter time of about 2 minutes to about 3 minutes.

Also, pathogenic bacteria that remain on a medical instrument and cause secondary infection can be effectively eliminated before the use of the medical instrument that can be reused, by performing sterilization complying with strict HLD standards on the medical instrument within a shorter time. Thus, the sterilization time for reuse of medical instruments such as an endoscope, a catheter inserted into a vessel, a trocar, and a surgery instrument can be minimized, and simultaneously, secondary infection can be prevented.

Furthermore, although a sterilization liquid is not completely removed after the sterilization of a medical instrument and the medical instrument is immediately used, the sterilization liquid does not irritate the mucous membranes of patient's lung, eyes, and nose, and patient's skin, thereby fundamentally preventing side effects caused by the sterilization of the medical instrument.

In addition, since sterilization complying with the HDL standards in which mycobacteria such as tubercle bacillus that is not easy to eliminate need to be exterminated by about 99.9999% can be achieved within a shorter time of about 2 minutes to about 3 minutes, corrosion of medical instruments formed of metallic materials during the sterilization process can be minimized.

### DESCRIPTION OF DRAWINGS

FIG. 1 is a diagram illustrating a sterilization apparatus for medical instruments according to an embodiment of the present disclosure;

FIG. 2 is a block diagram illustrating a configuration of an electrode lifespan limiting circuit;

FIG. 3 is a flowchart illustrating a sterilization method using the sterilization apparatus for the medical instrument of FIG. 1; and

FIG. 4 is a graph illustrating a Butler-Volmer equation showing a relationship between a current intensity and an overpotential of an electrode.

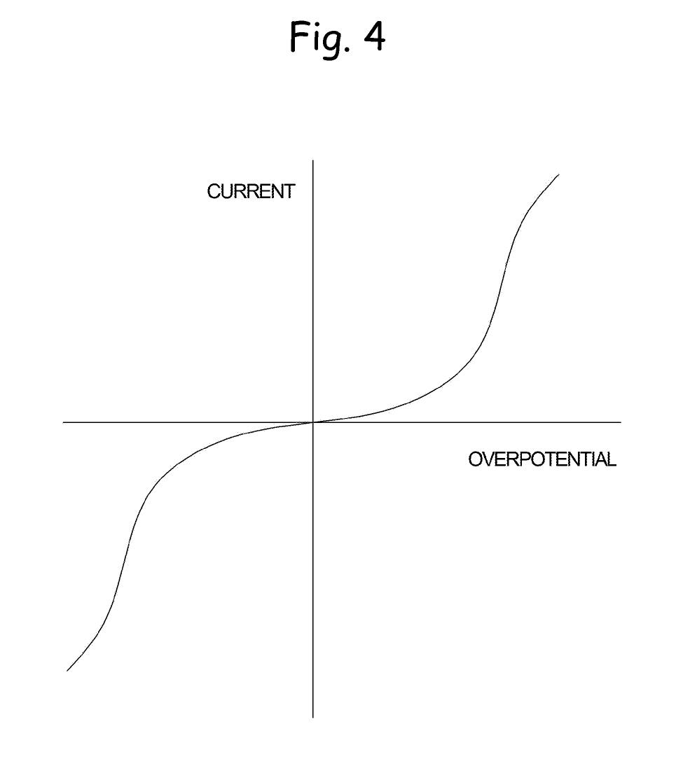

### BEST MODE

Hereinafter, exemplary embodiments will be described in detail with reference to the accompanying drawings. Throughout the drawings and the detailed description, unless otherwise described, the same drawing reference numerals will be understood to refer to the same elements, features, and structures. The relative size and depiction of these elements may be exaggerated for clarity, illustration, and convenience. The following detailed description is provided to assist the reader in gaining a comprehensive understanding of the methods, apparatuses, and/or systems described herein. Accordingly, various changes, modifications, and equivalents of the methods, apparatuses, and/or systems described herein will be suggested to those of ordinary skill in the art. Also, descriptions of well-known functions and constructions may be omitted for increased clarity and conciseness.

Hereinafter, an exemplary embodiment of a sterilization apparatus 100 for medical instruments will be described in detail with reference to the accompanying drawings.

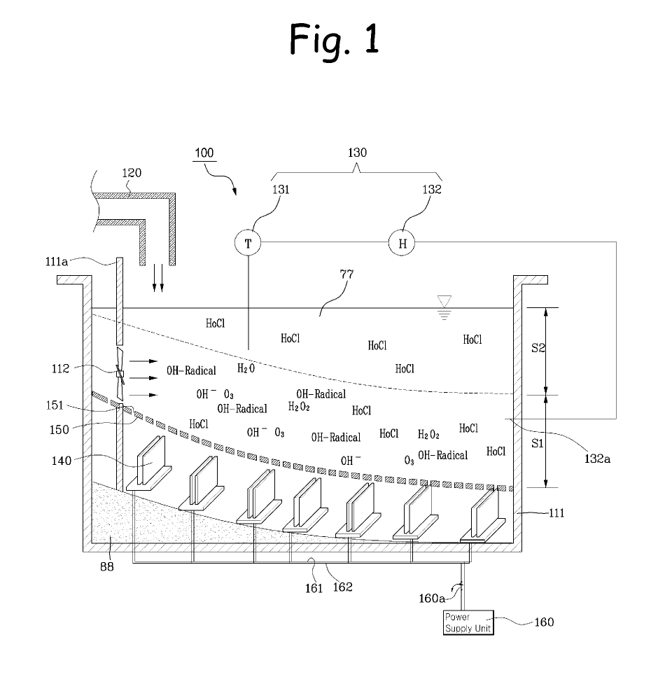

As shown in FIG. 1, the sterilization apparatus 100 for the medical instrument may include a container 111 for containing a solution containing chlorine, a saline solution supply unit 120 for supplying the saline solution that is heated to a temperature of about 60° C. or more, preferably, about 92.9° C. or more, a temperature control unit 130 for maintaining the solution of the container 111 at a temperature of about 60° C. or more, a plurality of electrodes 140 that are disposed on the bottom of the container 111, a medical instrument support 150 having a net shape with a plurality of holes to support the medical instrument, and a power supply unit 160 for supplying direct current to the plurality of electrodes 140.

The container 111 has a bottom 88 that is inclined to facilitate the placement of the medical instrument. The medical instrument support 150 is also inclined according to the inclination of the bottom 88 of the container 111. Thus, a part of medical instrument to be sterilized may be obliquely laid in the container 111 by a part to be inserted into a patient's body even when the medical instrument cannot be separated from an apparatus. The laid medical instrument is exposed to sterilizing components of free chlorine including hypochlorous acid, hydrogen peroxide (H₂O₂), OH radical, and ozone (O₃) that are generated from the plurality of electrodes 140 distributed over the bottom 88 of the container 111, the fat layer of mycobacteria is broken down by hot water, and then the medical instrument is sterilized within a shorter time by the sterilizing components of free chlorine, hydrogen peroxide (H₂O₂), OH radical, and ozone (O₃), which are generated by the electrolysis.

Particularly, based on many tests, the applicant of the present disclosure found that mycobacteria can be very effectively eliminated by the intermediate sterilizing components of hydrogen peroxide (H₂O₂), OH radical, and ozone (O₃) which are intermediate products existing only for a short time. However, since the intermediate products of the electrolysis, intermediate sterilizing components of hydrogen peroxide (H₂O₂), OH radical, and ozone (O₃) do not remain in the solution for a long time, excellent sterilization effect can be achieved only within a region S1 which is not far away from the electrode 140. Accordingly, although there is a difference according to the size of the electrode or the applied current, a target part (for example, part inserted into human body) of the medical instruments to be sterilized needs to be located within the region S1 near the electrode 140, rather than the region S2 distance from the electrode 140.

Although not shown in the drawing, the saline solution supply unit 120 may supply a solution in which water is preheated to a predetermined high temperature above 60° C. (preferably about 92.9° C. or more) and then mixed with chlorine such that chlorine ions remain. This prevents a limitation in which a desired sterilization effect is not shown because the concentration of chlorine is changed by the heating process, by separating chlorine from water through the heating process by the heater.

Although the solution is supplied to the container 111 at a temperature of about 60° C. or more through the saline solution supply unit 120, the solution may be naturally cooled by ambient temperature to be lowered to a temperature of about 60° C. or less. When the temperature of the solution is lowered to about 60° C. or less, the efficiency of deforming the cell water of the fat layer of mycobacteria may be reduced. Accordingly, the temperature control unit 130 measures in real-time the temperature of the solution 77 held in the container 111 by a thermometer 131, and controls the temperature of the solution 77 into a temperature range that can deform the cell wall of the fat layer of mycobacteria, by heating a hot wire 132a of the heater 132 based on the measurement result of the thermometer 131.

The electrode 140 may be configured with flat electrodes that face each other, and may be configured with various types of electrodes that are disclosed in Korea Patent Application Publication No. 2009-19639, filed by the applicant of the present disclosure. For example, the electrode 140 may have protrusions that face each other. Also, an electrode shown in FIG. 1 of the above patent gazette may be used, and the electrode may have a plurality of current carrying paths. However, since a medical instrument to be sterilized is easy to be exposed to a large amount of the sterilizing components of free chlorine, hydrogen peroxide (H₂O₂), OH radical, and ozone (O₃), the electrode 140 may be distributed over the bottom of the container 111.

The medical instrument support 150 has significantly large holes (for example, net shape) that allow connection to the electrode 140. Accordingly, the medical instrument support 150 may support the medical instrument such that sterilizing components of free chlorine, hydrogen peroxide (H₂O₂), OH radical and ozone (O₃) can contact the whole surface of the medical instrument. In this case, a fan 112 disposed on a partition 111a in the container 111 may forcibly flow the solution in the container 111, and thus the position of the medical instrument supported by the medical instrument support 150 may move to allow the sterilizing components of hydrogen peroxide (H₂O₂), OH radical and ozone (O₃) to contact the whole surface of the medical instrument.

The power supply unit 160 may supply direct current to the electrode 140 comprising a negative electrode and a positive electrode. In this case, the direct current may range from about 30 mA to about 2,000 mA. Also, a switch 160a may be disposed on positive and negative power supply lines 161 and 162 for supplying power to the electrode 140. The switch 160 may be turned on or off by electrical signals.

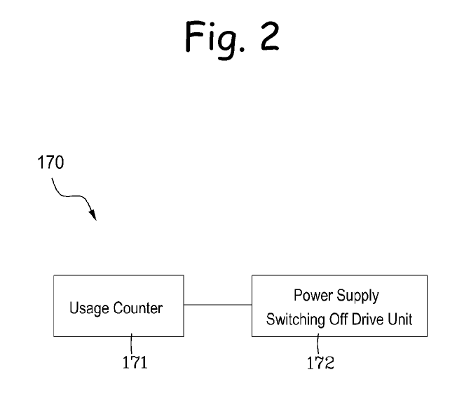

As shown in FIG. 2, the electrode use limiting unit 170 may include a usage counter 171 and a power supply switching off drive unit 172. The usage counter 171 counts the number of sterilization of a medical instrument. When a predetermined usage number is counted by the usage counter 171, power supply lines 161 and 162 for supplying power from the power supply unit 160 to the electrode 140 may be opened to allow power not to be supplied to the electrode 140 anymore.

Generally, platinum is plated on the electrode 140 to promote the electrolysis. In this case, as the electrolysis is repeated, the platinum on the surface of the electrode 140 is consumed. Finally, the amount of oxidant obtained may be reduced compared to the amount of oxidant obtained from the initial thickness of platinum. Even in this case, when the sterilization apparatus 100 normally operates, a user may wrongly think that the medical instrument is sufficiently sterilized, and then may use the medical instrument, causing secondary infection. Accordingly, the electrode use limiting unit 170 may enable reliable sterilization and washing of the medical instrument complying with HLD standards.

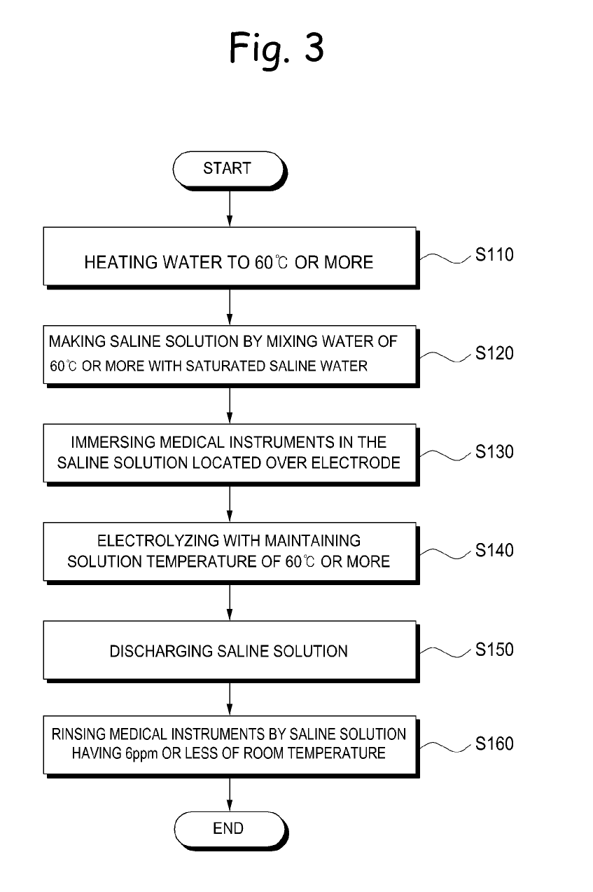

Hereinafter, a sterilization method for medical instruments according to an embodiment of the present disclosure will be described in detail with reference to FIG. 3.

Step 1

Although not shown, water to be used for sterilization and washing for medical instruments is heated to a temperature of about 60° C. or more (S110). In this case, water is heated to a temperature of about 93° C. to about 98° C. just before the boiling point in order to sterilize mycobacteria such as M. terrae having a strong fat layer water cell according to the diseases of patients for whom the medical is used.

Step 2

Thereafter, chlorine is mixed with water heated in Step 110 to prepare a saline solution containing chlorine ions (S120). A solution with a desired chlorine concentration may be prepared by mixing NaCl powder or saturated saline water with the water.

Step 3

The saline solution having a desired concentration (for example, concentration of physiological saline similar to the salt concentration of human body) prepared in Step 2 is supplied to the container 111 through the solution supply unit 120 shown in FIG. 1. When the saline solution 77 is sufficiently filled in the container 111, the medical instrument to be sterilized is placed on the medical instrument support 150. In this case, the medical instrument is located such that all parts of the medical instrument to be sterilized are located within the region 51 over the electrodes.

Step 4

Since the solution 77 of the container 111 is naturally cooled by heat exchange with ambient cold air after Step 3, the temperature of the solution 77 is always measured using a thermocouple thermometer having a fast response characteristics, and the saline solution 77 may be intermittently heated by the heater 132 such that the saline solution 77 is maintained within a predetermined temperature range (for example, about 75° C.). Thus, while the temperature of the solution 77 in the container 111 is maintained at a temperature of about 60° C. or more (for example, about 75° C.), a direct current is supplied from the power supply unit 160 to the electrode 140, and thus a large amount of sterilizing components of free chlorine, hydrogen peroxide (H₂O₂), OH radical, and ozone (O₃) are generated to sterilize mycobacteria.

In other words, when the fat layer cell wall surrounding mycobacteria is deformed and weakened by the high temperature solution, mycobacteria are disclosed to sterilizing components such as free chlorine and then the sterilizing components penetrate and kill the mycobacteria. Herein, hydrogen peroxide (H₂O₂), OH radical, and ozone (O₃) that play an important role in eliminating mycobacteria and are intermediate products of the electrolysis may effectively eliminate mycobacteria. Simultaneously, since proteins on the surface of the medical instrument are removed by the sterilizing components generated by the electrolysis, a washing effect as well as a sterilization effect can be achieved.

Here, when mycobacteria such as mycobacterium tuberculosis that is not easy to be killed are eliminated, it means that other microorganisms, bacteria, and viruses are substantially exterminated. Accordingly, with the above operation, all bacteria including mycobacteria stuck on the surface of the medical instrument may be eliminated by 6 Log₁₀ (99.9999%) in only two or three minutes, enabling sufficient sterilization complying with the HLD standards.

Step 5

since sufficient sterilization meeting the HLD requirements is performed on the medical instrument, the medical instrument that had been sterilized may be immediately used. However, a process for rinsing the medical instrument that had been sterilized may be performed. For this, the saline solution 77 used in sterilization is discharged from the container 111 (S150).

### Step 6

A saline solution of a room temperature, (heated saline solution is also possible to rinse) is put into the container 111, and then a low current of about 30 mA to about 150 mA and a DC voltage of about 2.2 V to about 3.4 V are applied to the electrode 140 so as to generate free chlorine of about 6 ppm or less in the electrode 140. Herein, it is also possible to use heated saline solution for the rinsing process. Then, a circulation fan 112 is rotated at a higher speed and thus to rinse and secondly sterilize the medical instrument using the free chlorine remaining in the solution 77 (S160). In this case, a method of generating free chlorine of about 6 ppm or less, disclosed in Korean Patent Application Publication No. 2009-19639, may be used.

Since the medical instrument is rinsed by a saline solution containing free chlorine of about 6 ppm or less instead of water, bacteria, viruses and the like that might be still stuck on the surface of the medical instrument may be eliminated. Also, since the solution contains free chlorine of about 6 ppm or less, even though the medical instrument is immediately inserted into a human body while contacting mucous membranes of the human body, the mucous membranes are not stimulated. Furthermore, since bacteria is eliminated by 99.9999%, a secondary infection of patients can be effectively prevented by the short-time sterilization and washing.

### INDUSTRIAL APPLICABILITY

A number of exemplary embodiments have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

1. A sterilization method for medical instruments, comprising:
preparing a solution containing chlorine and having a temperature of about 60° C. or more;
disposing at least one electrode in a container containing the solution and immersing at least one medical instrument in the solution such that the medical instrument is disposed over the electrode; and
electrolyzing the solution by applying a current to the electrode to generate sterilizing components of free chlorine comprising hypochlorous acid, hydrogen peroxide (H₂O₂), OH radical, and ozone (O₃) and thus sterilize the medical instrument by the sterilizing components which move up in the opposite direction of gravity from the electrode.

2. The sterilization method of claim 1, after the sterilizing of the medical instrument, further comprising:
discharging the solution; and
secondarily sterilizing the medical instrument by performing electrolysis such that a free chlorine comprising hypochlorous acid of about 6 ppm or less is generated.

3. The sterilization method of claim 1, further comprising applying a forcible flow such that a whole surface of the medical instrument contacts the solution in the container.

4. The sterilization method of claim 1, wherein the preparing of the solution comprises:
heating water to a temperature ranging from about 60° C. to about 90° C.; and
mixing the heated water with a saline component.

5. The sterilization method of claim 1, further comprising coating the electrode with platinum to form a platinum layer thereon, and shutting off the supplying of the current to the electrode when the platinum layer is consumed and worn out to be thinner than a predetermined thickness according to the use of the electrode.

6. The sterilization method of claim 1, wherein the solution is a saline solution.

7. The sterilization method of claim 1, wherein in the sterilizing of the medical instrument, the solution is maintained at a temperature of about 92.9° C. or more.

8. The sterilization method of claim 1, wherein the sterilizing of the medical instrument is performed while maintaining the solution at a temperature of about 60° C. or more.

9. A sterilization apparatus for medical instruments, comprising:
a heater for heating a liquid to a temperature of about 60° C. or more;
a container containing a solution containing the liquid with chlorine and receiving the medical instrument to be sterilized in the solution thereof;
a power supply unit for supplying a direct current;
an electrode disposed under the medical instrument so as to be immersed in the solution, electrolyzing the solution by supplying a direct current from the power supply unit to generate sterilizing components of free chlorine comprising hypochlorous acid, hydrogen peroxide (H₂O₂), OH radical, and ozone (O₃), and sterilizing the medical instrument by the sterilizing components which move up in the opposite direction of gravity from the electrode.

10. The sterilization apparatus of claim 9, wherein the heater is separate from the container.

11. The sterilization apparatus of claim 9, wherein the heater is disposed to heat the container, and the container is maintained at a temperature of about 60° C. or more.

12. The sterilization apparatus of claim 9, wherein a part of the medical instrument to be sterilized is immersed in the solution.

13. The sterilization apparatus of claim 9, wherein the container is disposed such that the medical instrument is laid therein, and the electrode is disposed under the medical instrument.

14. The sterilization apparatus of claim 9, wherein the electrode is coated with platinum, and when a life of the electrode ends, power is not applied to the electrode until the electrode is replaced.

15. The sterilization apparatus of claim 9, wherein the heater maintains the solution of the container at a temperature of about 92.9° C. or more.
